(19) United States
(12) Patent Application Publication
(10) Pub. No.: US 2005/0025738 A1
(43) Pub. Date: Feb. 3, 2005

(54) Liquid air freshener and water treatment for use with humidifiers

(76) Inventor: Chuck Haymond Hall, East Berlin, PA (US)

Correspondence Address: Chuck H. Hall, 600 Church Road, PO Box 853, East Berlin, PA, 17316, US

(21) Appl. No.: 10/628,728

(22) Filed: Jul. 28, 2003

(51) Int. Cl. A61L 9/00; A61L 9/01

(52) U.S. Cl. 424/76.1

(57) ABSTRACT

This invention is an all-in-one liquid solution, which provides air freshening and cleansing properties when used in various types of humidifiers.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND

The present invention is a liquid air freshener for use with various types of humidifiers. Specifically, this invention is an all-in-one liquid solution consisting of the following elements: a fragrance concentrate, water, color dye, anti-bacterial concentrate, and lime and calcium cleaning agent. The liquid solution benefits the user by providing a certain desirable scent to the immediate surrounding air, while at the same time providing a water treatment aiding in the cleaning of the humidifier.

### BRIEF SUMMARY OF THE INVENTION

This invention provides a desired fragrance through the use of humidification to the immediate surrounding environment while also providing a water treatment to aid in the cleaning of the user's humidifier. While water treatment products exist for the cleaning of humidifiers, none release fragrance, which gives a freshening effect to the immediate surrounding environment. In addition to the freshening of the immediate environment, certain fragrances are well known to provide aroma therapy, which may benefit the user with emotional, physical, and spiritual well being.

1. What I claim as my invention is: a liquid air freshener and water treatment for the use with humidifiers.
